(19) United States
(12) Patent Application Publication
(10) Pub. No.: US 2016/0245804 A1
(43) Pub. Date: Aug. 25, 2016

(54) Methods, Systems and Kits of Fecal NGal Rapid Immunochromatographic Test

(71) Applicant: Ping Gao, San Diego, CA (US)

(72) Inventor: Ping Gao, San Diego, CA (US)

(73) Assignee: Epitope Diagnostics, Inc., San Diego, CA (US)

(21) Appl. No.: 15/048,974

(22) Filed: Feb. 19, 2016

Related U.S. Application Data
(60) Provisional application No. 62/118,447, filed on Feb. 19, 2015.

(51) Int. Cl. G01N 33/543; G01N 33/68

(52) U.S. Cl. CPC G01N 33/54386; G01N 33/6893; G01N 2800/52; G01N 2800/065; G01N 2333/4704

(57) ABSTRACT

Disclosed are methods, systems and kits for rapid measurement of fecal NGal levels. The present invention provides a rapid measurement system with fecal sample collection, analyte extraction and immunochromatographic measurement of NGal levels, which can be used for diagnosing and monitoring gastrointestinal inflammation diseases such as chronic inflammatory bowel disease. In addition, the present invention provide a rapid test system for measuring different species of NGal, including total NGal, full-length NGal, C-terminal NGal fragments, N-terminal NGal fragments, and NGal fragments containing a specific region of an NGal molecule.

### CROSS-REFERENCES AND RELATED APPLICATIONS

This application claims priority to U.S. Provisional Application No. 62/118,447, entitled “Method, System and Kit of Fecal NGAL Rapid Immunochromatographic Test”, filed Feb. 19, 2015, the content of which is herein incorporated by reference in its entirety.

### BACKGROUND OF THE INVENTION

1. Field of the Invention

The present invention is in the field of immunotechnology. It particularly relates to methods, systems and kits for rapid measurements of fecal NGal.

2. Description of the Related Art

NGal (neutrophil gelatinase-associated lipocalin), also known as Lipocaline-2 (Lcn2) or oncogene 24p3, is a member of the lipocalin family of proteins that transport small, hydrophobic ligands. It is a pro-inflammatory factor mainly secreted by neutrophils and is highly inducible in response to various inflammatory stimuli. NGal plays an important role in innate immunity against bacterial infection by binding to bacterial siderophores, small iron-binding molecules, and preventing certain bacteria from acquiring iron. NGal may also function to transport irons and regulate cell growth and patterning in addition to its role in innate immunity. Most notably, the NGal level in serum and urine is significantly increased during the acute-phase response and in renal tubular injury, making it a sensitive biomarker for renal injury. Elevated level of NGal can be detected in urine sample before the occurrence of damages detectable by other methods, making NGal a good biomarker for early detection of kidney injury.

Many ELISA assays to measure NGal levels in blood and urine samples have been developed for detecting kidney disease and other diseases. For example, Barasch et al. use ELISA to measure NGal levels in urine or serum for diagnosis and monitoring of chronic renal disease (WO 2007047458). They use NGal cut-off values to assess mild, intermediate and advanced conditions of chronic renal disease. Mose et al. use NGal levels in urine samples as a biomarker to gauge the risk of having a cancer (US 20140242616) since NGal was found to be up-regulated in many cancers such as ovarian, colon, lung, and uterine cancer. In U.S. Pat. No. 8,313,919, Uttenthal et al. disclose a method of using the ratio of NGal levels in urinal vs. blood sample to distinguish renal injury from other events such as systemic inflammation, bacterial infection or cancers as the cause for NGal elevation. It is disclosed that the ratio of urinal vs. blood NGal higher than a pre-defined cut-off value can be indicative of acute renal injury or high risk of developing such injury.

Although many studies have focused on using NGal as a biomarker for renal diseases, recent studies (Oikonomou et al. J Gastroenterol. 2012, 47(5):519-30 & Ye [symbol] il et al. Digestive Diseases and Sciences. 2013, 58(9):2587-93.) have found that NGal is up-regulated in inflammatory bowel diseases (IBD) patients and serum NGal levels can be used as a biomarker for IBD diagnosis. Given that much of NGal produced by intestine is made by intestinal epithelial cells which secrete the majority into the lumen of intestine, Chassaing et al. (PloS ONE. 2012, 7(9):e44328) turned to the feces to look for NGal changes in gastrointestinal inflammatory diseases. They discovered that changes of fecal NGal, compared to serum NGal, provide a much more sensitive and specific means for detecting gastrointestinal inflammation. In addition, levels of fecal NGal are significantly increased during low-grade inflammation when it is hard to detect any changes of other pro-inflammatory biomarkers. The levels of fecal NGal are upregulated by over 10 folds and up to 10,000 folds in low-grade inflammation and robust inflammation models, respectively. Fecal NGal is therefore a sensitive and broadly dynamic biomarker for diagnosing and monitoring gastrointestinal inflammatory disease status.

Current method of measuring fecal NGal usually includes fecal sample collection, protein extraction and NGal detection by an immunoassay such as ELISA, immunoblotting or western blot. These types of conventional immunoassays are time consuming, and require expensive and complex machines which are usually not available to layperson or even professionals at small clinical settings. There is a need of a system for rapid NGal test that is easy to operate and can be used by a technician at a clinical laboratory, a nurse at doctor's office as well as a layperson at home.

### SUMMARY OF THE INVENTION

The present invention pertains to methods, systems and kits for rapid measurement of fecal NGal levels. It provides a rapid test system that includes fecal sample collection, analyte extraction and immunochromatographic measurement of NGal levels. The present invention provides a simple and sensitive chair-side test system for rapid determination of fecal NGal levels, which can be used for diagnosing and monitoring gastrointestinal inflammatory diseases such as chronic inflammatory bowel disease (IBD). In addition, the present invention provides a rapid test system for detecting different species of fecal NGals, including total NGal, full-length NGal, C-terminal NGal fragments, N-terminal NGal fragments and NGal fragments with a defined region, which provides a method for detecting a wide spectrum of NGal species that may have different clinical implications.

One embodiment of the invention provides a system for rapid measurement of different species of NGal in a fecal sample, comprising: a fecal sample collection structure, a sample extraction compartment and an NGal measurement compartment, wherein the NGal measurement compartment comprises an NGal test strip, wherein the NGal test strip comprises a conjugation pad deposited of a labeled NGal specific antibody and a test line immobilized with an NGal capturing antibody, wherein different combinations of the labeled NGal specific antibody and the NGal capturing antibody allow measurement of different species of NGal.

In one embodiment of the invention, the NGal measurement compartment houses an NGal immunochromatographic test strip that comprises an optional sample pad, a conjugation pad, an analysis membrane and an reservoir pad. The conjugation pad is disposed of an NGal-specific conjugate (mobile conjugate) that is labeled with a detectable marker. The analysis membrane comprises an NGal test line immobilized with an NGal capturing agent, which specifically binds to and captures free NGal and NGal bound with the labeled NGal-specific conjugates, and a control line disposed of immobilized agents that recognize and bind to the labeled NGal-specific conjugates, but not NGal itself. The immobilized compound at the control line will capture the labeled NGal-specific conjugates not retained by the capturing agents. In one embodiment, the system further includes a measuring device that can measure the intensity of signals from the detectable marker, and calculate the concentration of NGal by comparing the intensity value with a calibrated standard curve.

In one embodiment of the invention, the measurement range of fecal NGal is from 0.1 to 10,000 μg/g stool, and the preferred measurement range is 5 to 100 μg/g stool. The measurement time is 1 minute to 30 minutes after applying a test sample to the test strip, and the preferred measurement time is 5 minutes to 10 minutes.

One embodiment of the invention provides a rapid test system for measurement of total NGal species, wherein the conjugation pad comprises labeled anti-NGal antibodies capable of binding to all the different NGal species and the test line is immobilized with NGal capturing antibodies capable of binding to all the different NGal species, wherein the labeled anti-NGal antibody of the conjugation pad and the NGal capturing antibody at the test line can recognize different epitopes on an NGal species.

One embodiment of the invention provides a rapid test system for measurement of total NGal species, wherein the conjugation pad comprises labeled anti-NGal antibodies developed against full-length NGal protein and the test line is immobilized with NGal capturing antibodies developed against full-length NGal protein.

One embodiment of the invention provides a rapid test system for measurement of total NGal species, wherein the conjugation pad comprises labeled anti-NGal antibodies developed against an NGal fragment or multiple NGal fragments and the test line is immobilized with NGal capturing antibodies developed against an NGal fragment or multiple NGal fragments.

Another embodiment of the invention provides a rapid test system for detecting full-length NGal only, wherein the labeled anti-NGal antibody at the conjugation pad is a monoclonal or polyclonal antibody that specifically recognizes and binds to the N-terminal region of NGal protein, and the capturing agent at the test line of the analysis membrane is a monoclonal or polyclonal antibody that specifically recognizes and binds to the C-terminal region of an NGal protein, or vise versa. Using paired NGal N- and C-terminal specific antibody as the mobile conjugate and the NGal capturing agent leads to that only full-length NGal that binds to both the NGal N- and C-terminal specific antibody can be accumulated at the test line, thus detecting only full-length NGal in the fecal sample.

One embodiment of the invention provides a rapid test system for measurement of NGal fragments containing a specific region of NGal, wherein the conjugation pad comprises labeled antibody developed against the specific region of NGal and the test line is immobilized with NGal capturing antibodies capable of recognizing all the different epitopes on an NGal molecule.

One embodiment of the invention provides a rapid test system for detecting C-terminal NGal fragments, wherein the conjugation pad is disposed of labeled NGal antibodies that specifically recognize and bind to the C-terminal region of NGal, and the test line of the analysis membrane is immobilized with polyclonal NGal antibodies that are developed against full-length NGal protein and can bind to different epitopes of an NGal molecule. Another embodiment of the invention provides a rapid test for measuring the N-terminal NGal fragments, wherein the conjugation pad is disposed of labeled NGal antibodies that specifically recognizes and binds to N-terminal region of NGal, and the test line of the analysis membrane is disposed of immobilized polyclonal NGal antibodies that are developed against full-length NGal protein and bind to different epitopes of an NGal molecule.

One embodiment of the invention provides a rapid test system for simultaneously detecting full-length and C-terminal fragments of NGal, wherein the NGal test strip comprises a conjugation pad disposed of labeled NGal antibodies that specifically recognizes and binds to C-terminal region of NGal, a first test line of the analysis membrane immobilized with NGal antibodies that specifically recognize and bind to N-terminal region of NGal, and a second test line of the analysis membrane immobilized with NGal polyclonal antibodies that are developed against full-length NGal protein and bind to different epitopes on NGal. The quantity of full-length and C-terminal fragments of NGal can be determined by measuring the label intensity at the first test line and the second test line, respectively. Another embodiment of the invention provides a rapid test system for simultaneously detecting full-length and N-terminal fragments of NGal, wherein the NGal test strip comprises a conjugation pad disposed of labeled NGal antibodies that specifically recognize and bind to N-terminal region of NGal, a first test line of the analysis membrane immobilized with NGal antibodies that specifically recognize and bind to C-terminal region of NGal, and a second test line of the analysis membrane immobilized with NGal polyclonal antibodies that are developed against full-length NGal protein and bind to different epitopes on NGal. The quantity of full-length and N-terminal fragments of NGal can be determined by measuring the label intensity at the first test line and the second test line, respectively.

One embodiment of the invention provides a system for rapid measurement of fecal NGal comprising a fecal sample collection structure, a sample extraction compartment and an NGal measurement compartment. The sample extraction compartment reversibly houses the fecal sample collection structure, contains sample extraction solution, and has a breakable seal on one end of the sample extraction compartment. The NGal measurement compartment comprises an NGal test strip, a piercing structure and a rigid container. The piercing structure of the NGal measurement compartment can break through the breakable seal of the sample extraction compartment so as to allow fluid communication between two compartments. Once the sample extraction solution is in contact with the NGal test strip, the rapid NGal test can be performed on the NGal test strip. In a preferred embodiment, the fecal sample collection structure is integrated with the cap of the sample extraction compartment. The breakable seal of the sample extraction compartment is a plastic film that covers an aperture opposite to the sample collection structure. The NGal measurement compartment can be attached to the sample extraction compartment by puncturing the breakable seal using the piercing structure in a “push-in” or “screw-in” mode.

One embodiment of the invention provides a kit for rapid measurement of NGal in a fecal sample, comprising: a fecal sample collection structure, a sample extraction compartment, an NGal measurement compartment, and an instruction manual, wherein the NGal measurement compartment comprises an NGal test strip, wherein the NGal test strip comprises a conjugation pad deposited of labeled NGal specific antibodies and a test line immobilized with an NGal capturing antibody, wherein different combinations of the labeled NGal specific antibody and the NGal capturing antibody allow measurement of different species of NGal, and wherein the sample extraction compartment comprises a fecal sample extraction solution. The instruction manual provides a step-by-step guidance to use the rapid measurement system. To quantitatively determine the fecal NGal, the kit further includes a label measuring device that can read, calculate and display the concentration of NGal by measuring the intensity of the label and comparing the intensity value with a calibrated standard curve. As described above, the labeled NGal specific antibody at the conjugation pad and the NGal capturing antibody can be configured to detect different species of NGals including total NGal, full-length NGal and NGal fragments containing a specific region of NGal.

One embodiment of the invention provides a method for diagnosing inflammatory bowel diseases in a patient suspected of having inflammatory bowel diseases, comprising: a) measuring NGal level in a patient's fecal sample using the rapid NGal measurement kit above; b) comparing patient's NGal level with those of healthy people, wherein it is indicative of inflammatory bowel diseases in the patient if patient's fecal NGal level is significantly higher than those of healthy people.

One embodiment of the invention provides a method for monitoring the progress of treatment of inflammatory bowel disease in a patient, comprising: a) measuring NGal levels in patient's fecal sample using the rapid NGal measurement kit above over a time period after or during the treatment of the disease; b) plotting the fecal NGal level over the measurement time period, wherein a upward trend of NGal levels indicates deteriorating condition of the disease and a downward trend of NGal levels indicates improving condition of the disease.

### BRIEF DESCRIPTION OF THE DRAWINGS

FIG. 1. Components of the rapid fecal NGal measurement system. A, an immunochromatographic test strip with the following elements: 1, a sample pad; 2, a conjugation pad; 3, a test line; 4, a control line; 5, a reservoir pad; 6, an analysis membrane; and 7, a backing sheet. B, a sample extraction compartment with a sample collection structure and sample extraction solution. C, an NGal measurement compartment having an immunochromatographic test strip housed in a cassette, which has an opening for sample loading and a window for observing the test line and the control line. D, an NGal measurement compartment having an immunochromatographic test strip in the form of a dipstick.

FIG. 2. Exemplary configuration of different NGal test strips.

FIG. 3. Interpretation of NGal rapid test results.

FIG. 4. An embodiment of a 3-in-1 rapid fecal NGal measurement system, which has a sample extraction compartment having a sample collection structure, a breakable seal and a sample extraction solution, and an NGal measurement compartment having an NGal test strip and a piercing structure.

FIG. 5. Another embodiment of the 3-in-1 rapid fecal NGal measurement system, which has a sample extraction compartment with a breakable seal, and an NGal measurement compartment with a piercing structure.

### DETAILED DESCRIPTION

Measuring and monitoring NGal levels in fecal samples is a valuable tool for diagnosing gastrointestinal inflammatory disease and monitoring the progression of the disease. The present invention provides a rapid and easy-to-use fecal NGal test system that combines fecal sample collection, protein extraction and immunochromatographic measurement of different species of NGal. This rapid NGal test requires no expensive machinery, is easy to operate, and can be used by a skilled technician in a clinical laboratory, a nurse in a doctor's office as well as a layperson at home. Another advantage of the test is the fast speed and high sensitivity. The test can be performed within 5 to 10 minutes and has a measurement range from 0.1 to 10,000 μg NGal per gram of stool. In addition, The rapid NGal test system of the invention can detect different NGal species, including total NGal, full-length NGal, and NGal fragments containing a specific region of NGal.

### General Definitions

Unless defined otherwise, all technical and scientific terms used herein have the same meaning as commonly understood by an artisan of ordinary skills in the field to which this invention belongs. The following terms are defined below for the sake of clarity and ease of reference.

All references to the plural herein shall also mean the singular and to the singular shall also mean the plural unless the context otherwise requires.

The term “NGal”, as used herein, refers to neutrophil gelatinase-associated lipocalin, also known as Lipocaline-2 (Lcn2) or oncogene 24p3, which is a member of the lipocalin family of proteins that transport small, hydrophobic ligands. NGal is encoded by the LCN2 gene in human. NGal is highly expressed in neutropils and is used as a biomarker for acute kidney injury and inflammatory bowel diseases.

The term “NGal fragment”, as used herein, refers to a continuous portion of an NGal molecule. An NGal fragment can be a portion of NGal shorter than the full-length NGal. It also includes the full-length NGal, the largest NGal fragment. The term “C-terminal NGal fragment” or “C-terminal NGal peptide”, as used herein, refers to an NGal fragment containing an intact C-terminal, which includes the full-length NGal. The term “N-terminal NGal fragment” or “N-terminal NGal peptide”, as used herein, refers to an NGal fragment containing an intact N-terminal, which includes the full-length NGal.

The term “species of NGal”, as used herein, refers to different forms of NGal existing in a physiological sample, e.g. urine, blood or fecal sample. NGal secreted from neutrophils is subjected to proteinase digestion. Physiological samples are likely to contain NGal fragments with different lengths, including full-length NGal and shorter fragments of NGal, for example, C-terminal NGal fragments, N-terminal NGal fragments, and NGal fragments lacking C-terminal and/or N-terminal. Different chemical modification of NGal, e.g. phosphorylation of NGal, is also considered to be a different form of NGal, that is, a different species of NGal.

The term “total NGal”, as used herein, refers to all the existing forms of NGal in a sample, including full-length NGal and all the shorter NGal fragments.

The term “NGal N-terminal specific antibody”, as used herein, refers to a monoclonal or polyclonal antibody or antigen binding fragments that specifically recognize and bind to the N-terminal region of NGal (e.g. a N-terminal region with at least 5, 10 or 20 amino acids). The NGal N-terminal specific antibody can be developed by immunizing animals with short N-terminal peptides (e.g. 5, 10, or 20 amino acid N-terminal peptide) or by immunizing animals with a larger N-terminal peptide (e.g. 40, 60, or 80 amino acid N-terminal peptide) and purifying the antibody with a shorter N-terminal peptide (e.g. a 7 amino acid N-terminal peptide).

The term “NGal C-terminal specific antibody”, as used herein, refers to a monoclonal or polyclonal antibody or antigen binding fragments that specifically recognize and bind to the C-terminal region of NGal (e.g. a C-terminal region with at least 5, 10 or 20 amino acids). The NGal C-terminal specific antibody can be developed by immunizing animals with short C-terminal peptides (e.g. 5, 10, or 20 amino acid C-terminal peptide) or by immunizing animals with a larger C-terminal peptide (e.g. 40, 60, or 80 amino acid C-terminal peptide) and purifying the antibody with a shorter C-terminal peptide (e.g. a 7 amino acid C-terminal peptide).

The term “labeled NGal specific antibody”, as used herein, refers to an NGal specific antibody conjugated with a detectable marker. The detectable marker is a chemical moiety emitting a signal that is detectable to human eyes and/or can be detectable by a machine. For example, the detectable marker includes, but not limited to, colored dyes, fluorescent dyes, radioactive compound and microparticles composed of gold, magnetic materials, polystyrene, silica, acrylic or the like. The NGal specific antibody can be polyclonal or monoclonal antibody and antigen binding fragments that specifically recognizes and associates with NGal or NGal fragments. Either the whole antiserum, the affinity purified antibody to NGal, the IgG purified fraction, or the antigen binding fragments (Fab or F(ab′)₂) of the antibody, may be employed.

The present invention provides a system for rapid measurement of different species of NGal in a fecal sample, comprising: a fecal sample collection structure, a sample extraction compartment and an NGal measurement compartment, wherein the NGal measurement compartment comprises an NGal test strip, wherein the NGal test strip comprises a conjugation pad deposited of a labeled NGal specific antibody and a test line immobilized with an NGal capturing antibody, wherein different combinations of labeled NGal specific antibody and NGal capturing antibody allow measurement of different species of NGal.

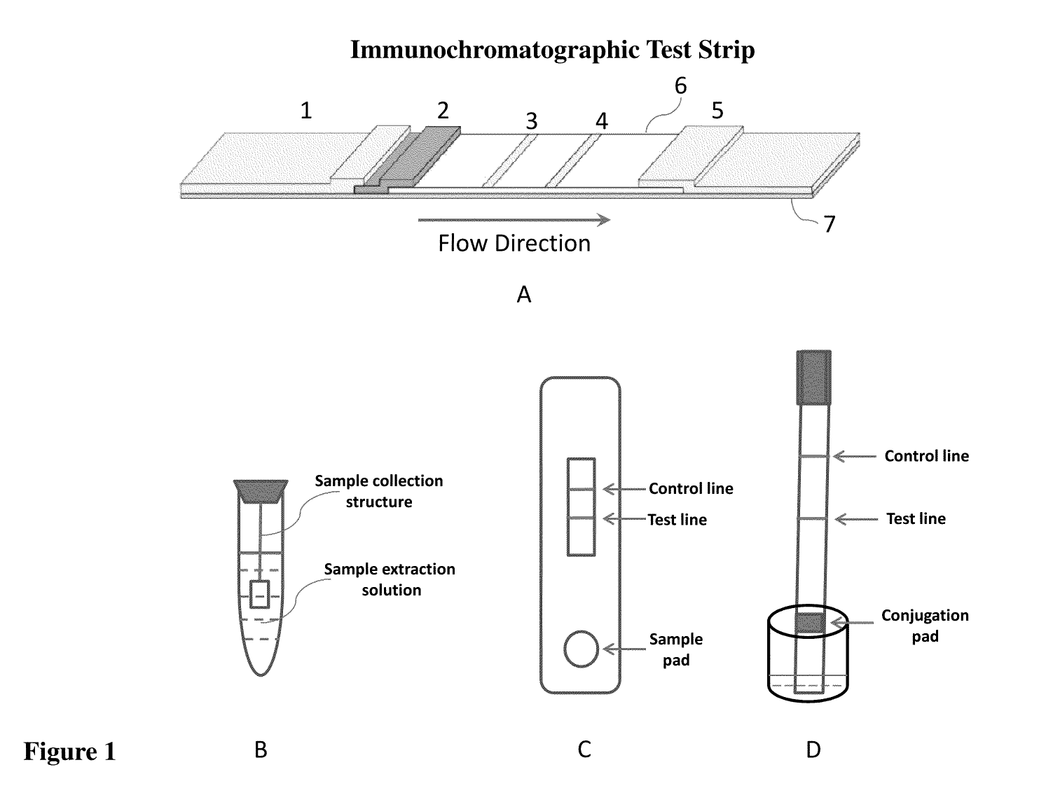

The sample extraction compartment houses the fecal sample collection structure and contains a sample extraction solution that can substantially dissolve the fecal sample and extract proteins in the sample (FIG. 1B). The fecal sample collection structure is preferably integrated to a lid or cap of the sample extraction compartment, which can form a fluid-tight seal. The lid or cap may be a screw cap or a snap cap made of plastic or other suitable material. The fecal sample collection structure can be any suitable structure for collecting or obtaining a solid fecal sample, including, but not limited to, a tube, a spatula, a cross-shaped column, a wand, and the like. The fecal sample collection structure can also be any suitable structure for collecting a liquid fecal sample such as a pipette. The sample extraction solution is a buffered salt solution used to suspend and dissolve fecal samples and extract proteins from the fecal sample. The sample extraction solution may include sodium chloride and/or sodium phosphate such as PBS (phosphate buffered solution) or Tris-hydrochloride at a certain pH range (e.g. pH 6.0 to 9.5). The sample extraction solution may also include one or more sugars, preservatives and detergents like tween-20 and triton X-100.

The NGal measurement compartment comprises an NGal measurement means, preferably an immunochromatographic NGal test strip (FIG. 1A). The NGal test strip can be used as a dipstick (FIG. 1D) or housed in a cassette or other suitable container (FIG. 1C). The suitable container is preferably made of transparent/semitransparent materials or has a transparent window for viewing the measurement result. The container also has an open slot for access of a sample solution. The container may be of any desirable shape, for example, tubular or rectangular.

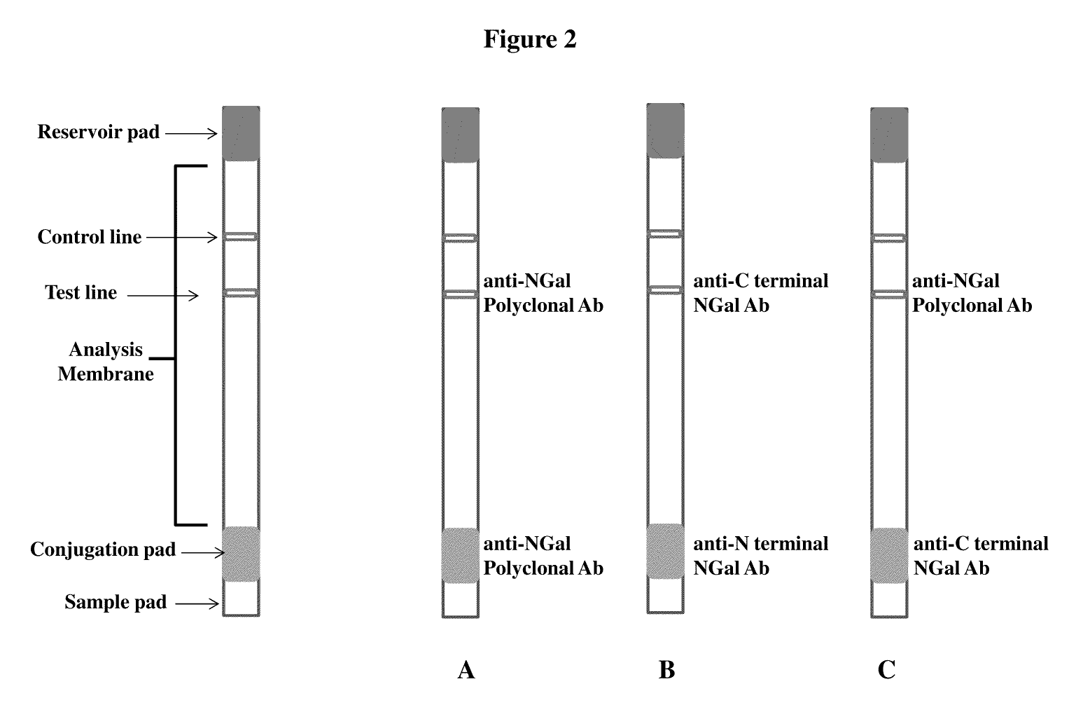

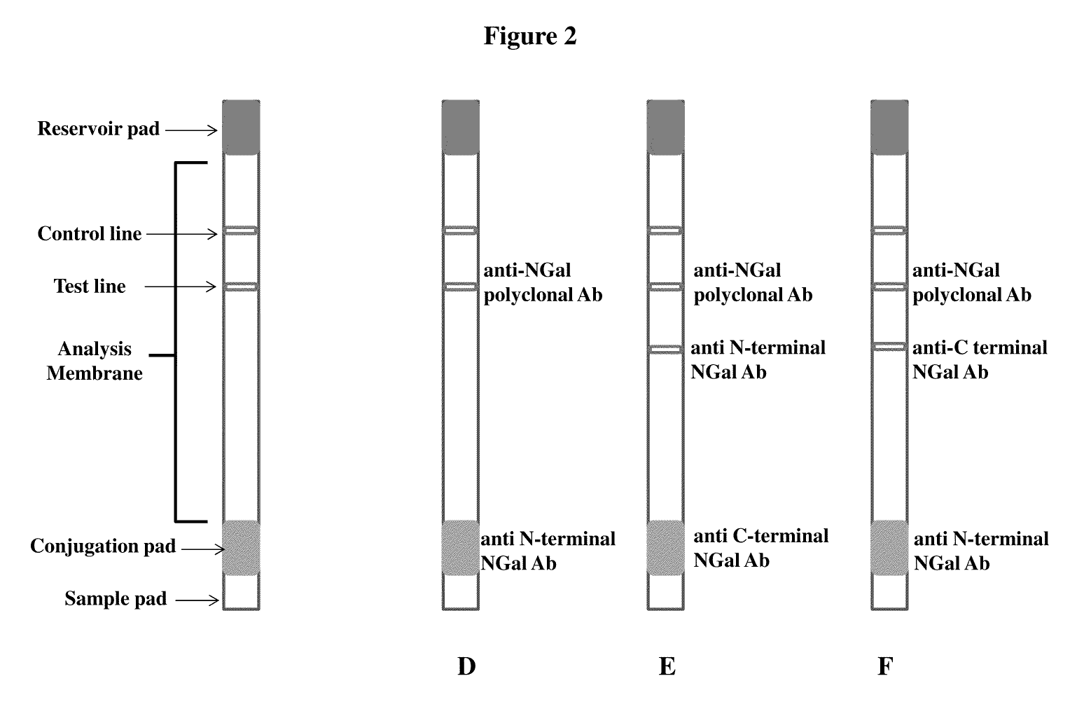

The NGal test strip from the bottom to the top comprises an optional sample pad, a conjugation pad, an analysis membrane, and an optional reservoir (FIG. 2). The sample pad is the first element of the test strip, comprised of absorptive materials to absorb sample solution and drive the flow of sample solution into the test strip. The reservoir pad, the last element of the test strip, is composed of absorptive materials that facilitates the uptake of liquid running through the test strip and provides additional wicking volume. The conjugation pad is a porous membrane evenly disposed of labeled conjugates that specifically recognizes and binds to NGal such as anti-NGal antibody or other NGal binding compounds. The conjugation pad is preferably made of low or non-protein binding materials such as glass fiber. The labeled NGal-specific conjugates once dissolved in a buffered solution can migrate with the buffer along the analysis membrane. Therefore the labeled NGal-specific conjugates in the conjugation pad is also called the mobile conjugate. The anti-NGal antibodies can be prepared in immunized animals such as rabbits, sheep, goats, rats, mice or other immunized species of animals, by monoclonal antibody techniques, antibody cloning technologies, or other antibody production technologies known to those skilled in the art. Either the whole antiserum, the affinity purified antibody to NGal, the IgG purified fraction, or the antigen binding fragments (Fab or F(ab′)₂) of the antibody, may be employed. The methods for immunization of animals and the preparation and purification of antibody is performed according to standard laboratory procedures and known to those skilled in the art. The label on the mobile antibody conjugate is a detectable chemical moiety including, but not limited to, colored dyes, fluorescent dyes, or microparticles composed of gold, magnetic materials, polystyrene, silica, acrylic or the like. Preferably, the label is composed of colored dyes, colloidal gold or the like that can be visible to human eyes.

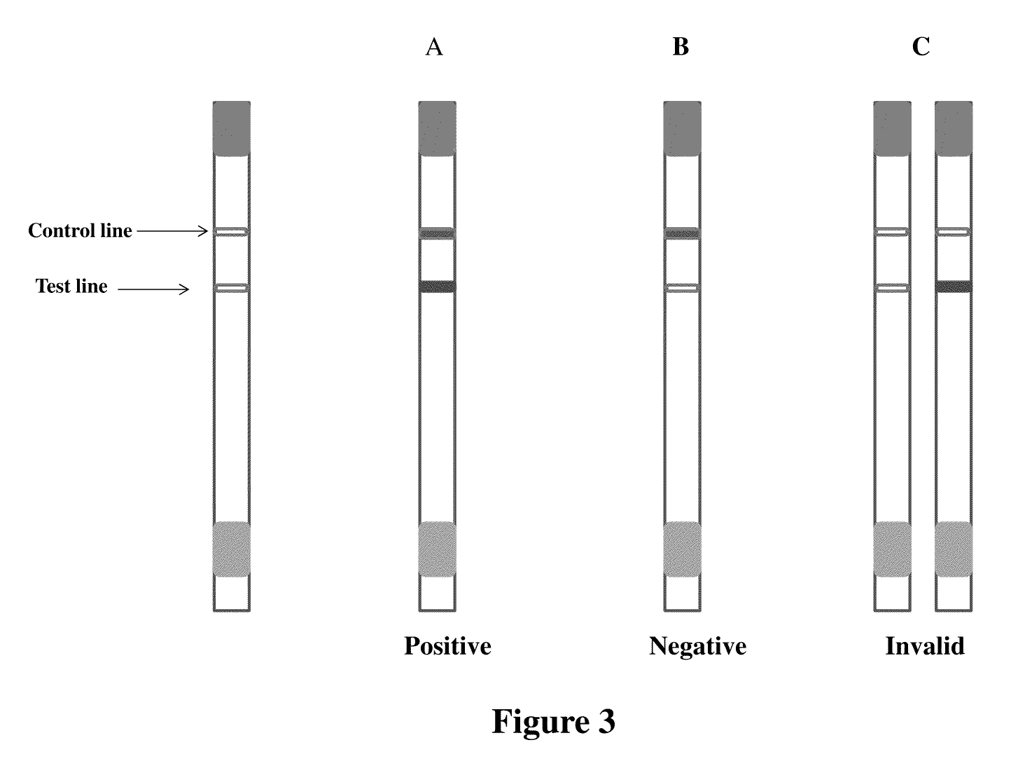

The analysis membrane is a lateral flow membrane to which analytical reagents for capturing NGal in the test sample are immobilized. It is a porous membrane made of materials such as, but not limited to, nitrocellulose or cellulose acetate. The analysis membrane has at least one test line, optionally followed by a control line. The test line comprises NGal specific capturing agents that are immobilized to the membrane, which is used to capture NGal bound with the labeled NGal-specific conjugate. The NGal specific capturing agents may be anti-NGal antibodies that bind to NGal on a different binding site other than that of the labeled conjugates in the mobile phase. The detection of positive signals at the test line indicates the presence of NGal in the test sample. The control line comprises an immobilized compound that binds to the labeled NGal-specific conjugates which are not captured at the test line. The immobilized compound at the control line may be a species-specific anti-immunoglobulin antibody that recognizes and captures the labeled antibodies not captured by the test line. For example, a goat anti-mouse antibody can capture all the labeled mouse antibodies. Alternatively, the control line capturing compound could be immobilized NGal or NGal connected to an inert carrier protein such as bovine serum albumin. The immobilized NGal can capture all the labeled anti-NGal antibody that are not captured at the test line. The detection of the positive signals at the control line confirms the validity of the assay. For a qualitative test, detection/observation of label signals at the test line and the control line of the analysis membrane indicates the presence of NGal in the fecal sample (FIG. 3A); detection of label signals at the control line only, but not at the test line, indicates the absence of NGal in the fecal sample (FIG. 3B); and no detection of signals at the control line indicates an invalid test (FIG. 3C). For a quantitative test, a measuring device/machine is used to read, calculate and display the concentration of NGal by measuring the intensity of the label and comparing the intensity value with a calibrated standard curve. The measuring device used will depend upon the type of detection marker that is used to label the NGal. For example, a fluorometer, a spectrometer and a reflectance spectrophotometer will be used for measurement of fluorescent labels, colored labels and colloidal gold labels, respectively.

To perform the rapid measurement of fecal NGal, use the sample collection structure to collect sufficient amount of fecal sample and suspend the collected fecal sample in the extraction solution. Mix well the fecal sample and the extraction solution to ensure a thorough solubilization and extraction of the sample. Spin down insoluble pieces and add the supernatant of the extracted solution to the sample pad of the NGal test strip of the measurement compartment. If there is NGal present in the sample, NGal first forms a complex with the labeled NGal specific antibody in the conjugation pad, and the complex migrates along the analysis membrane and is captured by immobilized NGal capturing agents at the test line, showing a positive test line. The unbound labeled NGal specific antibodies are not captured at the test line and continue to travel to the control line where they are captured by immobilized capturing compounds that specifically recognize the labeled NGal specific antibodies, showing a positive control line. If both the test line and the control line are positive, this indicates that NGal is present in the fecal sample of the test (FIG. 3A). If only the control line is positive and the test line is negative, this indicates that NGal is absent from the fecal sample of the test (FIG. 3B). If the control line is negative, the performed test is not valid (FIG. 3C). Alternatively, the intensity of label on the test line can be measured by a suitable measuring device, and the concentration of NGal can be calculated by comparing the intensity value with a calibrated standard curve.

In one embodiment, the present invention provides a system for measurement of total NGal that includes full-length NGal and all the shorter NGal fragments existing in a sample. In this configuration (FIG. 2A), the conjugation pad is deposited with labeled anti-NGal antibodies capable of binding to all the species of NGal and the test line is immobilized with NGal capturing antibodies capable of binding to all the species of NGal, wherein the labeled anti-NGal antibody of the conjugation pad and the NGal capturing antibody at the test line can recognize different epitopes on an NGal species.

Antibodies capable of binding to all the species of NGal may be polyclonal or monoclonal antibodies. For example, polyclonal antibodies developed against full-length NGal protein, which include antibodies recognizing all the different epitopes of NGal, can be employed for this purpose. Antibodies capable of binding to total NGal species may also be polyclonal antibodies developed against an NGal fragment or multiple NGal fragments. In some cases, full-length protein may not be the best choice to induce generation of antibodies in an animal. An NGal fragment or multiple NGal fragments may be used to immunize animals and NGal specific polyclonal antibodies from immunized animals are collected to make a pool of antibodies that can recognize all the different epitopes on NGal. Alternatively, monoclonal antibodies can be developed against different epitopes of NGal. A collection of monoclonal antibodies that recognize all the different epitopes of NGal can also be employed in this configuration. During an immunochromatographic test, all the NGal species form complexes with labeled NGal specific antibodies at the conjugation pad. When the complexes of NGal-labeled NGal specific antibody migrate to the test line, they will be captured by the NGal capturing antibodies at the test line that recognize different epitopes on the complexes. Using this configuration, total NGal species in a sample can be measured.

In another embodiment of the invention, the rapid test can be designed to detect full-length NGal only. In this configuration, NGal C-terminal specific antibody and NGal N-terminal specific antibody are paired up to serve as either the labeled anti-NGal antibody in the mobile phase or the NGal capturing antibody at the test line. For example, if the NGal C-terminal specific antibody is used as the labeled mobile conjugate, the NGal N-terminal specific antibody is used as the NGal capturing agent. On the other hand, if the NGal N-terminal specific antibody is used as the labeled mobile conjugate, the NGal C-terminal specific antibody is used as the NGal capturing agent (FIG. 2B). Under this configuration, only the full-length NGal with intact C-terminal and N-terminal can form a labeled antibody-NGal-capturing antibody complex that can be detected at the test line. NGal fragments missing either N-terminal or C-terminal region will not form such a complex, and cannot be detected at the test line. Techniques to develop NGal N-terminal specific antibody is known to one skilled in the art. For example, the NGal N-terminal specific antibodies can be developed by immunizing animals with a first N-terminal peptide or a first N-terminal peptide linked to a protein carrier (e.g. bovine serum albumin, keyhole limpet hemocyanin, and ovalbumin). The N-terminal specific antibodies can then be obtained by affinity purification against a second N-terminal peptide. The first and second N-terminal peptide can be the same peptide or different ones. For example, the first N-terminal peptide can be aa1-10, aa1-15, aa1-20, or aa1-40 N-terminal peptides of NGal, and the second N-terminal peptide can be aa1-5 or aa1-7 N-terminal peptides. By using shorter N-terminal peptides to purify antibodies, it ensures that antibodies that specifically bind to the most distal portion of the N-terminal of NGal are isolated for this application. The C-terminal specific anti-NGal antibodies can be developed in a similar manner.

In one embodiment, the present invention provides a system for measurement of NGal fragments containing a specific region of NGal, wherein the conjugation pad comprises labeled antibody developed against the specific region of NGal and the test line is immobilized with NGal capturing antibodies capable of recognizing all the different epitopes on an NGal molecule. This system is useful for measuring NGal fragments with a specific region of interest. It employs a labeled NGal antibody that specifically recognizes the region of interest to form complexes with NGal fragments containing the specific region. The complexes can be captured and detected at the test line by the NGal capturing antibody.

In another embodiment of the invention, the labeled mobile conjugate is an NGal C-terminal specific antibody and the NGal capturing agent of the test line is polyclonal or monoclonal anti-NGal antibodies that recognize all the different epitopes of NGal (FIG. 2C). Under this configuration, only the NGal fragments with intact C-terminal will be bound with labeled antibody and be detected as positive at the test line.

In another embodiment of the invention, the labeled mobile conjugate is an NGal N-terminal specific antibody and the NGal capturing antibody of the test line is a polyclonal or monoclonal anti-NGal antibodies that recognize all the different epitopes of NGal (FIG. 2D). Under this configuration, only the NGal fragments (including full-length NGal) with intact N-terminal will bind with the labeled antibody and be detected as positive at the test line.

In another embodiment of the invention, there are two test lines embedded in the analysis membrane, allowing simultaneous measurement of full-length and C-terminal or N-terminal fragments of NGal. In one embodiment, the labeled mobile conjugate is an NGal C-terminal specific antibody and the NGal capturing antibody of the first test line is an NGal N-terminal specific antibody and the second test line comprises polyclonal or monoclonal anti-NGal antibodies that recognize all the different epitopes of NGal (FIG. 2E). Under this configuration, the NGal species with intact C-terminal first form a complex with the labeled NGal C-terminal specific antibody. The labeled complexes migrate along the analysis membrane until they meet immobilized NGal N-terminal specific antibody at the first test line. Those labeled complexes having NGal with both intact C-terminal and intact N-terminal regions are captured by NGal N-terminal specific antibody at the first test line. The remaining NGal-labeled NGal conjugate complexes continue to travel and will be captured by antibodies at the second test line. By measuring the label intensity at the first and second test line, the amount of full-length and C-terminal fragments in the sample can be calculated.

In another embodiment, the labeled mobile conjugate is an NGal N-terminal specific antibody and the NGal capturing antibody of the first test line is an NGal C-terminal specific antibody and the second test line comprises polyclonal or monoclonal anti-NGal antibodies that recognize all the different epitopes of NGal (FIG. 2F). Under this configuration, the NGal fragments with intact N-terminal first form a complex with the labeled NGal N-terminal specific antibody. The NGal-labeled NGal specific conjugate complexes migrate along the analysis membrane until they meet the immobilized NGal C-terminal specific antibody at the first test line. Those labeled complexes having a full-length NGal are captured by NGal N-terminal specific antibody at the first test line. The remaining labeled complexes continue to travel and will be captured by antibodies at the second test line. By measuring the label intensity at the first and second test line, the amount of full-length and N-terminal fragments in the sample can be calculated.

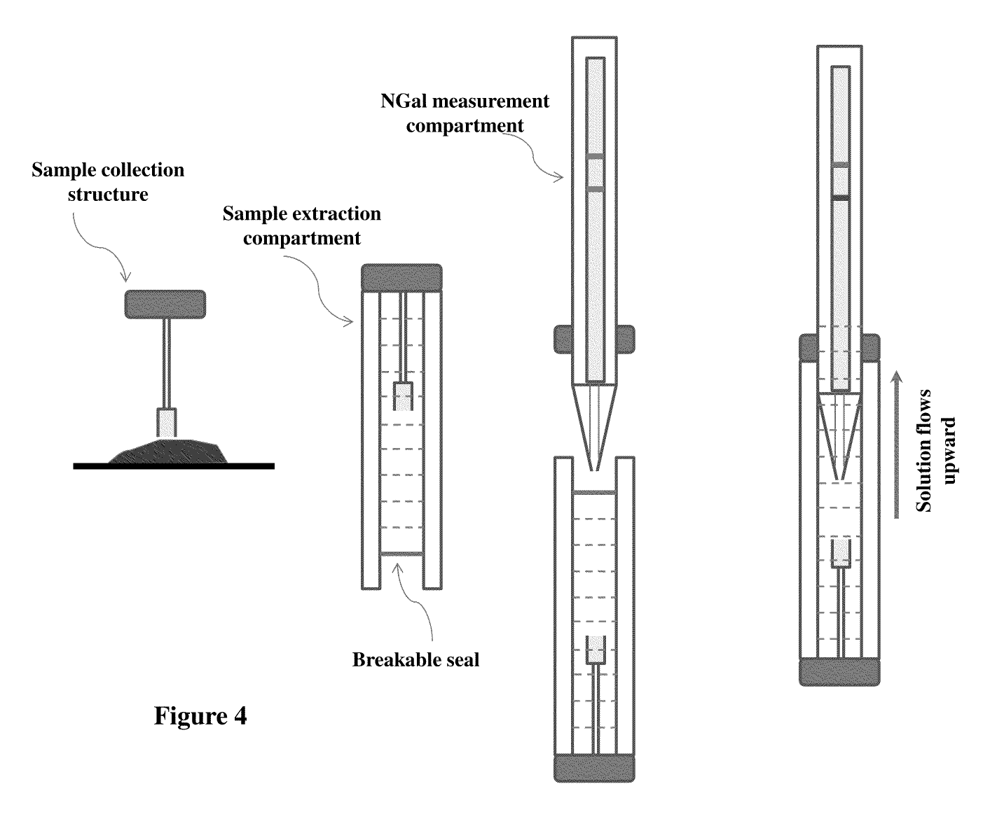

The present invention provides a 3-in-1 system for rapid measurement of fecal NGal that can be performed in an essentially closed unit after fecal sample collection. This system can minimize cross-contamination between samples and contamination to the environment and people performing the test. The rapid test system comprises a fecal sample collection structure, a sample extraction compartment and an NGal measurement compartment (FIGS. 4 & 5). The sample extraction compartment reversibly houses a fecal sample collection structure, contains a sample extraction solution, and have a breakable seal on one end of the sample extraction compartment. The fecal sample collection structure is preferably integrated to a lid or cap of the sample extraction compartment. The sample extraction compartment includes a breakable seal preferably at the end opposite to the sample collection structure-connected lid. The breakable seal may be flush with an end or be recessed within the extraction compartment, where it forms a fluid-tight seal with the extraction compartment. The breakable seal may be made of any puncturable wrap or film capable of holding aqueous solutions including, but not limited to, plastic wrap, plastic film and aluminum wrap.

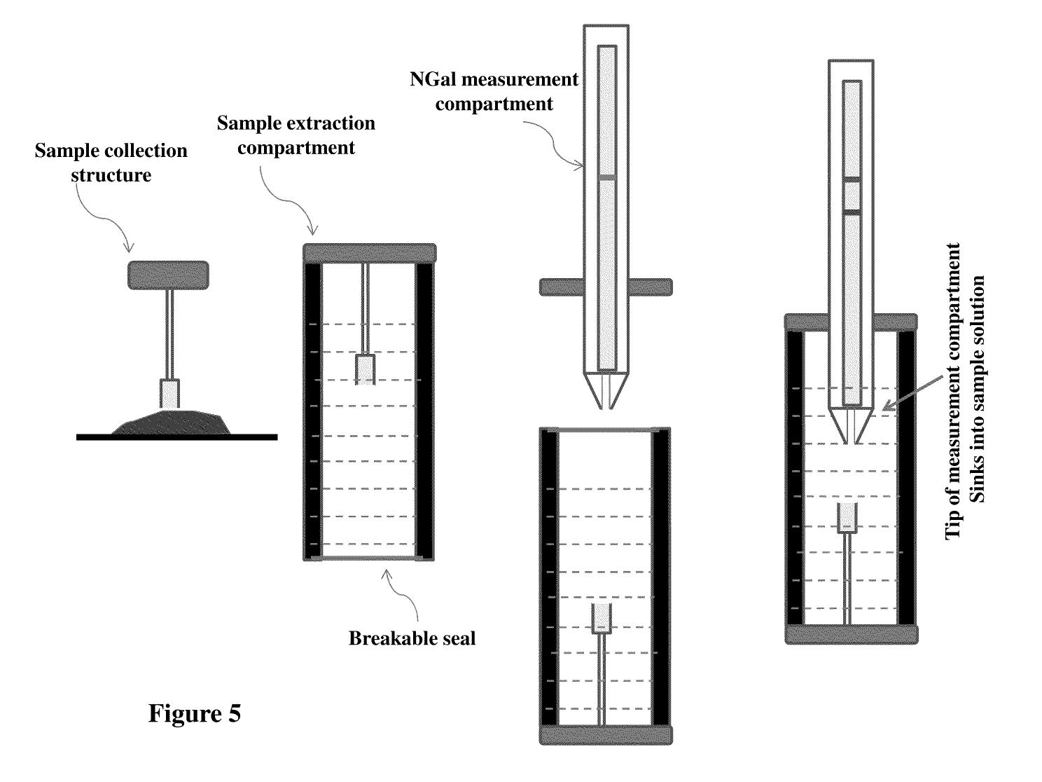

The NGal measurement compartment comprises an NGal test strip, a piercing structure and a rigid container. The rigid container houses the NGal test strip and is connected to the piercing structure. The rigid container is preferably made of transparent or semitransparent materials such as polystyrene or polypropylene, which allows viewing of the NGal measurement means without opening the container. The rigid container may be of any desirable shape, for example, tubular or rectangular. The piercing structure has a sharp end that allows it to puncture through the breakable seal of the sample extraction compartment. In one embodiment, the piercing structure is shaped to be complementary to the shape of the breakable end of the sample extraction compartment. The insertion of the piercing structure into the sample extraction compartment displaces part of the sample extraction solution, causing the displaced solution to flow from the sample extraction compartment into the measurement compartment (FIG. 4). In another embodiment, the NGal test strip is positioned close to the end of the piercing structure. Breaking through the breakable seal, the piercing structure is inserted deep enough into the sample extraction solution such that it allows the bottom of the NGal test strip to be immersed into the sample extraction solution (FIG. 5).

To perform the rapid NGal assay using the 3-in-1 system above, unscrew the lid with the sample collection structure and insert the sample collection structure into at least 2 different spots of a stool specimen to collect sufficient amount of stool sample. Insert the sample collection structure back into the sample extraction compartment. Mix well the collected fecal sample with the sample extraction solution to ensure complete suspension and solubilization of the sample. Let the sample extraction compartment stand with the breakable seal side facing up for about one minute to settle down large sample pieces or the sample extraction compartment can be briefly centrifuged to spin down the large pieces. Insert or screw down the piercing structure of the measurement compartment into the sample extraction compartment through the breakable seal to connect these two compartments. The sample extraction solution flows into the measurement compartment and get access to the NGal test strip, allowing NGal species in the sample to be detected by the NGal test strip. If both the test line and the control line are positive, this indicates that NGal is present in the fecal sample of the test. If only the control line is positive and the test line is negative, this indicates that NGal is absent from the fecal sample of the test. If the control line is negative, the performed test is not valid. Alternatively, the intensity of label on the test line can be measured by a suitable label reader, and the concentration of NGal can be calculated by comparing it with a calibrated standard curve.

One embodiment of the invention provides a kit for rapid measurement of NGal in a fecal sample, comprising: a fecal sample collection structure, a sample extraction compartment, an NGal measurement compartment, and an instruction manual, wherein the NGal measurement compartment comprises a immunochromatographic NGal test strip, wherein the NGal test strip comprises a conjugation pad deposited of labeled NGal specific antibodies and a test line immobilized with an NGal capturing antibody, wherein different combinations of labeled NGal specific antibody and NGal capturing antibody allow measurement of different species of NGal, and wherein the sample extraction compartment comprises a fecal sample extraction solution. The sample extraction solution may include sodium chloride and/or sodium phosphate such as PBS (phosphate buffered solution) or Tris-hydrochloride at a certain pH range (e.g. pH 6.0 to 9.5). The sample extraction solution may also include one or more sugars, preservatives and detergents like tween-20 and triton X-100. The instruction manual provides a step-by-step guidance to use the rapid measurement system. To quantitatively determine the fecal NGal, the kit further includes a measuring device that can read, calculate and display the concentration of NGal by measuring the intensity of the label and comparing the intensity value with a calibrated standard curve. As described above, the labeled NGal specific antibody at the conjugation pad and the NGal capturing antibody immobilized at the test line can be configured to detect different species of NGal including total NGal, full-length NGal and NGal fragments containing specific regions of NGal.

NGal is a small secreted protein highly expressed in neutrophils. Human NGal has been reported to increase in patients with inflammatory bowel diseases. As gastrointestinal inflammation leads to increased secretion of NGal into feces, Fecal NGal is an early and sensitive biomarker for diagnosing gastrointestinal inflammatory diseases. The present invention provides a method for diagnosing inflammatory bowel diseases in a patient suspected of having inflammatory bowel diseases, comprising: a) measuring the NGal level in a patient's fecal sample using the rapid NGal measurement kit above; b) comparing patient's NGal level with those of healthy people, wherein it is indicative of inflammatory bowel diseases in the patient if patient's fecal NGal level is significantly higher than those of healthy people. The NGal level measured herein could be, for example, the total NGal or the full-length NGal.

Tested in mice models for intestinal inflammation, levels of fecal NGal increased 10 folds during the low-grade inflammation while they are further increased to 10,000 folds in robust inflammation models. These results suggest that levels of fecal NGal is a good biomarker to track the severity of intestinal inflammation. The present invention also provides a method for monitoring the progress of treatment of inflammatory bowel disease in a patient, comprising: a) measuring NGal levels in patient's fecal sample using the rapid NGal measurement kit above over a time period after or during the treatment of the disease; b) plotting the fecal NGal level over the measurement time period, wherein a upward trend of NGal levels indicates deteriorating condition of the disease and a downward trend of NGal levels indicates improving condition of the disease. The NGal level measured herein could be, for example, the total NGal or the full-length NGal, whichever demonstrates a good correlation with the progress of the disease.

### EXAMPLES

### Example 1

### Preparation of Antibodies Specific For N-Terminal Portion and C-Terminal Portion of NGal

An N-terminal portion of human NGal peptide is used to immunize animal for the production of either polyclonal antibody or a monoclonal antibody. The preferred amino acid sequence of the N-terminal NGal is “MPLGLLWLGL ALLGALHAQA QDSTSDLIPA PPLSKVPLQ”. This peptide is produced via chemical peptide synthesis or as a recombinant protein expressed in a E. coli cell line. The corresponding NGal N-terminal specific antibody binds to both the above N-terminal NGal peptide and the NGal protein/fragments having the N-terminal peptide.

A C-terminal portion of human NGal peptide is used to immunize animal for the production of either polyclonal antibody or a monoclonal antibody. The preferred amino acid sequence of the C-terminal NGal is “GRTKELTSEL KENFIRFSKY LGLPENHIVF PVPIDQCIDG”. This peptide is produced via chemical peptide synthesis or as a recombinant protein which may be expressed in an E. coli cell line. The corresponding NGal C-terminal specific antibody binds to both the above C-terminal NGal peptide and the NGal protein/fragments having the C-terminal peptide.

### Example 2

### Preparation of Polyclonal Antibody That Recognizes Different Epitopes on NGal

Recombinant full-length NGal protein is expressed in E. coli, CHO or HEK239 cell lines. The purified protein is used to immunize a sheep to produce anti-NGal antiserum. Polyclonal anti-NGal antibody is further purified from the antiserum via affinity purification using an NGal-resin column or a Protein A or protein A/G column. This polyclonal anti-NGal antibody can bind to different epitopes on NGal and can be used to make labeled NGal-specific mobile conjugate or capturing antibody immobilized to the test line of the analysis membrane.

### Example 3

### Preparation of NGal Test Strip For Measurement of Total NGal

Colloidal gold conjugated sheep anti-NGal polyclonal antibody raised from animals immunized with full length NGal protein is diluted with a highly purified water based solution which may include bovine serum albumin and sucrose, to an OD 10 at 520 mn on a spectrophotometer. This conjugated antibody is directly spread onto a 32 mm width glass fiber (for example, Millipore G028 glass fiber) to make a conjugation pad. The conjugation pad is dried in an oven.

An unlabeled sheep anti-NGal polyclonal antibody is diluted to a concentration from 0.5 mg/ml-2.5 mg/ml in a 0.01M Phosphate Buffer Saline and striped onto the lower 1/2 part (the test line) of an analysis membrane made of nitrocellulose (for example, Millipore HF-135). A rabbit anti-sheep antibody is also diluted to a concentration of 1.0 mg/ml and striped onto the upper ½ part (the control line) of the same membrane. The analysis membrane is dried in an oven immediately.

The analysis membrane, the conjugation pad and a paper based absorption pad is laminated with a supporting or backing card and cut into 70 mm long and 3.5 mm width strips. This test strip is assembled into the NGal measurement compartment.

### Example 4

### Preparation of NGal Test Strip For Measurement of Full-Length NGal

To prepare an NGal test strip for measurement of full-length NGal, an anti-N-terminal NGal specific monoclonal or polyclonal antibody is fixed onto the test line of the analysis membrane and the colloidal gold conjugated anti-C-terminal NGal specific antibody is placed onto the conjugation pad. The antibody configuration may also work vice versa.

### Example 5

### Preparation of NGal Test Strip For Simultaneous Measurement of Full-Length NGal and C-Terminal Fragments

To prepare an NGal test strip for simultaneous measurement of full-length NGal and C-terminal fragments, there are two test lines fixed on the analysis membrane. The conjugation pad is deposed of the colloidal gold conjugated C-terminal NGal specific antibody. The first test line is fixed with an N-terminal NGal specific antibody and the second test line is fixed with an anti-full length NGal polyclonal antibody.

### Example 6

### Procedure of Fecal NGal Measurement Using the Rapid NGal Test System

The rapid NGal test system comprises a sample extraction tube which has a sampling lid connected to a sample collection structure, and a measurement tube which has a piercing tip and houses an NGal test strip.

### Specimen Collection

Collect solid specimen: Unscrew the sampling lid integrated with the sample collection structure and keep the sample extraction tube in a vertical position to prevent the loss of the extraction solution (0.01M phosphate buffer saline, 0.1% BSA, 0.1% Tween-20, 0.05% NaN₃, pH 7.4). Insert and twist the tip of the sample collection structure into the stool specimen at two or more different sites. Collect fecal sample that is stuck to the sample collection structure. Do not intentionally collect any separate and large pieces of fecal sample. Insert the sample collection structure into the sample extraction tube and secure the sampling lid tightly.

Collect liquid specimen: Unscrew the sampling lid and keep the sample extraction tube in a vertical position to prevent the loss of extraction solution. Using a plastic single use pipette to collect three drops of the liquid stool sample and add to the sample extraction tube. Place the sampling lid back into the tube and secure it tightly.

### Assay Procedure

Thaw and bring collected specimens to room temperature (8-30° C.) if needed. Mix the sample extraction tube vigorously to ensure a good liquid suspension and complete solubilization of the fecal sample. Position the sample extraction tube with the breakable seal side facing up and let it sediment for about 1 minute. Alternatively, the sample extraction tube can be centrifuged briefly to spin down large fecal pieces. Screw the measurement tube in a vertical position into the sample extraction tube by breaking through the breakable seal of the sample extraction tube. Tighten the screw securely so that no liquid can be leaked from the connection. Allow the extraction solution flow into the bottom space of the measurement tube and contact the bottom of the NGal test strip and keep the device in a vertical position. Read test result between 5 to 10 minutes after the extraction solution is in contact with the NGal test strip. The test result is positive if both the test line and the control line are positive. The test result is negative if only the control line is positive and the test line is negative. If the control line is negative, the performed test is invalid.

### Performance Evaluation

The sensitivity and specificity of this NGAL test device were studied with 206 clinical samples and compared with an NGal ELISA test. The data are shown as below.

| NGal | ELISA | | |
|---|---|---|---|
| Rapid Test | Positive | Negative | Total |
| Positive | 68 | 2 | 70 |
| Negative | 2 | 134 | 136 |
| Total | 70 | 136 | 206 |

### Specificity: 98.5% (134/136)

### Sensitivity: 97.1% (68/70)

### Accuracy: 98.1% (202/206)

Inter-series and intra-series accuracy: 100%

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

### REFERENCES

1. Chassaing B, Srinivasan G, Delgado M A, Young A N, Gewirtz A T, Vijay-Kumar M. Fecal Lipocalin 2, a Sensitive and Broadly Dynamic Non-Invasive Biomarker for Intestinal Inflammation. PloS ONE. 2012, 7(9):e44328.
2. Oikonomou KA1, Kapsoritakis A N, Theodoridou C, Karangelis D, Germenis A, Stefanidis I, Potamianos S P. Neutrophil gelatinase-associated lipocalin (NGAL) in inflammatory bowel disease: association with pathophysiology of inflammation, established markers, and disease activity. J Gastroenterol. 2012, 47(5):519-30.
3. Ye [symbol] il Al, Gönen C, Senate [symbol] E, Paker N, Gökden Y, Ko [symbol] han K, Erdem E D, Güildüz F. Relationship between neutrophil gelatinase-associated lipocalin (NGAL) levels and inflammatory bowel disease type and activity. Digestive Diseases and Sciences. 2013 Sep; 58(9):2587-93.

What is claimed:

1. A system for rapid measurement of different species of NGal in a fecal sample, comprising: a fecal sample collection structure, a sample extraction compartment and an NGal measurement compartment, wherein the NGal measurement compartment comprises an NGal test strip, wherein the NGal test strip comprises a conjugation pad deposited of a labeled NGal specific antibody and a test line immobilized with an NGal capturing antibody, wherein different combinations of the labeled NGal specific antibody and the NGal capturing antibody allow measurement of different species of NGal.

2. The system of claim 1 for quantitative measurement of NGal further comprises a measuring device.

3. The system of claim 1, wherein the measurement range of NGal is 0.1 to 10,000 μg/g fecal sample.

4. The system of claim 1, wherein the operation time for performing the rapid measurement of NGal ranges from 1 to 30 minutes.

5. The system of claim 1, wherein the NGal measurement compartment comprises an immunochromatographic test strip.

6. The system of claim 1 for measurement of total NGal, wherein the conjugation pad comprises labeled anti-NGal antibodies capable of binding to all the species of NGal and the test line is immobilized with capturing antibodies capable of binding to all the species of NGal, wherein the labeled anti-NGal antibody of the conjugation pad and the NGal capturing antibody at the test line can recognize different epitopes on an NGal species.

7. The system of claim 6, wherein the conjugation pad comprises labeled anti-NGal antibodies developed against full-length NGal protein and the test line is immobilized with NGal capturing antibodies developed against full-length NGal protein.

8. The system of claim 6, wherein the conjugation pad comprises labeled anti-NGal antibodies developed against an NGal fragment or multiple NGal fragments and the test line is immobilized with NGal capturing antibodies developed against an NGal fragment or multiple NGal fragments.

9. The system of claim 1 for measurement of full-length NGal, wherein the conjugation pad comprises labeled NGal N-terminal specific antibody paired with the test line immobilized with NGal C-terminal specific antibody, or the conjugation pad comprises labeled NGal C-terminal specific antibody paired with the test line immobilized with NGal N-terminal specific antibody.

10. The system of claim 1 for measurement of NGal fragments containing a specific region of NGal, wherein the conjugation pad comprises labeled antibody developed against the specific region of NGal and the test line is immobilized with NGal capturing antibodies capable of recognizing all the different epitopes on NGal.

11. The system of claim 1, wherein the sample extraction compartment reversibly houses the fecal sample collection structure and has a breakable seal; and wherein the NGal detecting compartment further comprises a piercing structure that can break the breakable seal to connect the sample extraction compartment and the NGal measurement compartment.

12. A kit for rapid measurement of different species of NGal in a fecal sample, comprising: a fecal sample collection structure, a sample extraction compartment, an NGal measurement compartment and an instruction manual, wherein the sample extraction compartment comprises a fecal sample extraction solution; wherein the NGal measurement compartment comprises an NGal test strip, wherein the NGal test strip comprises a conjugation pad deposited of labeled NGal specific antibodies and a test line immobilized with an NGal capturing antibody, wherein different combinations of the labeled NGal specific antibody and the NGal capturing antibody allow measurement of different species of NGal.

13. A method for diagnosing inflammatory bowel disease in a patient suspected of having inflammatory bowel disease, comprising the steps of:
a, measuring NGal level in a patient's fecal sample using the rapid measurement kit of claim 12;
b, comparing patient's NGal level with those of healthy people, wherein it is indicative of inflammatory bowel diseases in the patient if patient's fecal NGal level is significantly higher than those of healthy people.

14. A method for monitoring the progress of treatment of inflammatory bowel disease in a patient, comprising the steps of:
a, measuring NGal levels in patient's fecal sample using the rapid measurement kit of claim 12 over a time period after or during the treatment of the disease;
b, plotting the fecal NGal level over the measurement time period, wherein an upward trend of NGal levels indicates deteriorating condition of the disease and a downward trend of NGal levels indicates improving condition of the disease.
